(19) United States
(12) Patent Application Publication
(10) Pub. No.: US 2007/0203500 A1
(43) Pub. Date: Aug. 30, 2007

(54) Apparatus and method of shaping an intervertebral space

(75) Inventors: Jeffrey David Gordon, Saratoga Springs, NY (US); John K. Song, Chicago, IL (US)

Correspondence Address: John K Song, #4508, 474 North Lakeshore Drive, Chicago, IL, 60611, US

(73) Assignee: Vermillion Technologies, LLC, Chicago, IL (US)

(21) Appl. No.: 11/711,277

(22) Filed: Feb. 27, 2007

Related U.S. Application Data
(60) Provisional application No. 60/777,271, filed on Feb. 28, 2006.

(51) Int. Cl. A61F 2/44; A61B 17/17; A61B 17/16

(52) U.S. Cl. 606/96; 623/17.11; 606/80

(57) ABSTRACT

An apparatus and method for creating a space of defined length, height, width and shape with a guided mill in preparation for receiving a spinal implant or graft of known size and configuration is disclosed.

### CROSS-REFERENCE TO RELATED PATENT APPLICATION

This application claims priority to and the benefit of, pursuant to 35 U.S.C. §119(e), U.S. provisional patent application Ser. No. 60/777,271, filed Feb. 28, 2006, entitled “Apparatus and method of shaping an intervertebral space” by Jeffrey David Gordon and John K Song and is incorporated herein by reference in its entirety.

### FIELD OF THE INVENTION

The present invention generally relates to a surgical device for creating a cavity between or within bones of the human body.

### BACKGROUND OF THE INVENTION

Spinal surgery is a rapidly expanding field and interbody grafts (grafts placed between two adjacent vertebrae) are an important means of supporting the space between the vertebrae for purposes of fusion or motion preservation.

To implant an interbody graft, whether in the cervical, thoracic, or lumbar spine, an anterior approach to the spine is often performed. The space where the implant (e.g. cage, spacer, vertebral body replacement, bone dowel, arthoplasty device, etc.) is to be placed is most commonly prepared by hand with simple tools (such as a drill, curettes, osteotomes, etc). However, this can leave gaps between the bones and the implant resulting in sub-optimal results.

Numerous methods exist for preparing an exact cavity between or within adjacent bones for accepting an implant. Cloward described a technique whereby a drill is used to create a cylindrical cavity partially within the disc space and overlapping the vertebral bodies above and below (Cloward R B, Am J Surg, 1959). This technique has been widely used throughout the world. More recently, Michaelson (REFXXXXXX) described a means of guiding a drill while distracting the disc space.

We present a device which is placed into the interbody space and maintains distraction while providing a means of guiding a cutting tool. This is different from the prior art in that a guide post, not a tube, is utilized. In addition, the device will also provide for an automatic depth stop and prevent the cutting tool from penetrating too far and potentially damaging the delicate nervous structures immediately behind the disc space.

### SUMMARY OF THE INVENTION

The present invention is directed to an apparatus and method for use in spinal surgery for creating a space of selected shape and dimensions across the disc space between two adjacent vertebrae of the spine with a guided mill. The present invention comprises instrumentation and a surgical method of preparing vertebral endplates for the procedure, be it fusion or non-fusion, and specifically the creation of a space of a known shape and dimensions. The foregoing is achieved by the use of a mill which is guided by a novel guide mechanism. The instrumentation of the present invention allows for the safe, controlled and protected preparation of the disc space to the optimal depth and width. The present invention allows for the maximum stability of the graft/implant, as well as the construct, by providing for the greatest possible interface surface area and congruency between the graft/implant and each of the adjacent vertebrae.

It is an object of the present invention to provide for a surgical method and instrument means for performing interbody spinal fusion or in the alternative of inserting an “artificial disc implant” for the purpose of maximizing the width and optimizing the depth of the disc and the bone removed from front to back, or, back to front, from the vertebral endplates adjacent the disc space to be fused or implanted while confining such bone resection safely within the lateral, anterior (front) and posterior (back) limits of the disc space.

It is another object of the present invention to provide for a surgical method and instrument means for performing interbody spinal fusion or “artificial disc” implantation that provides for the rapid creation of both a known surface contour of each of the vertebral endplates adjacent to a disc space as well as a known and reproducible shape of the fusion or implantation site itself.

It is another object of the present invention to provide for a surgical method and instrument means for performing interbody spinal fusion that allows for the utilization of a larger interbody spinal fusion implant(s) than was possible with the prior art, such an implant having the capacity for providing increased amounts of osteogenic material, increased surface area, increased area of contact, increased stability and the ability to provide for greater support through the fusion or bone ingrowth area.

It is another object of the present invention to provide for a surgical method and instrumentation for performing the preparation of the space between adjacent vertebrae for the purpose of implanting an artificial disc or fusion implant(s) having the optimal cross sectional area of contact with said adjacent vertebrae and where said cross sectional area may be as large as possible while remaining safely within the perimeter of the endplates of the adjacent vertebrae.

It is a further object of the present invention to create a counterbore for the insertion of an implant which incorporates tabs into the space between adjacent vertebrae so that the implant sits substantially flush with the front of the vertebrae.

It is a further object of the present invention to describe means to create a counterbore for the insertion of an implant which incorporates tabs into the space between adjacent vertebrae with a spring loaded counterboring tool which adapts for use with multiple implant sizes.

The following is a brief outline of the steps of the surgical method of the present invention describing the use of the specific instrumentation in regard to the preferred embodiment:
1. The appropriate area of the spine is exposed and a partial disectomy is performed, whereby a portion and preferably a large portion of the disc is removed while preserving the annulus fibrosis portion of the disc along at least one side of the disc space.
2. The interspace so created may be distracted and while not requisite, preferably to its optimal height, which height is determined by the known normal spatial relationships for that area and the adjacent soft tissue -structures. The interspace is then measured for height, depth, and width. The width of the interspace may be determined in reference to the inferior portion of the vertebral endplate of the superior vertebrae, and this determines the selection of the appropriate width for the guide mechanism. The measured depth of the interspace, that is the distance between the front and back of vertebrae, will also determine the selection of a guide mechanism. The height and depth of the interspace will determine the selection of the appropriately sized mill.
3. The guide mechanism includes a spacer portion to separate the vertebral bodies and create a space in the intervertebral region of appropriate height. The width and depth of bone resection may then be easily confirmed visually prior to any actual bone resection.
4. The properly dimensioned mill is then guided by the guiding mechanism into the disc space in the appropriate orientation.
5. The mill is inserted into the disc space and the space is then milled to remove a portion of bone from the endplates adjacent to the disc space.
6. The prepared space may be irrigated and suctioned and then the mill is removed.
7. The guide mechanism is then removed and the appropriate implant or implants are then inserted into the prepared space.

### BRIEF DESCRIPTION OF THE DRAWINGS

FIG. 1 is an exploded, perspective view of the guide mechanism and insertion handle of the present invention.

FIG. 2 is a perspective view of the guide mechanism and insertion handle of the present invention.

FIG. 3 is an exploded, perspective view of the mill mechanism of the present invention

FIG. 4 is a perspective view of the mill mechanism of the present invention.

FIG. 5 is a perspective view of the guide mechanism and insertion handle assembly of the present invention showing insertion into a disc space between two vertebrae.

FIG. 6 is a perspective view of the guide mechanism and insertion handle assembly of the present invention after insertion into a disc space between two vertebrae.

FIG. 7 is a perspective view of the guide mechanism and insertion handle assembly of the present invention showing removal of the insertion handle after insertion of the guide mechanism into a disc space between two vertebrae.

FIG. 8 is a perspective view of the guide mechanism and milling mechanism assembly of the present invention showing guided insertion into a disc space between two vertebrae.

FIG. 9 is a perspective view of the guide mechanism and milling mechanism assembly of the present invention shown after insertion into a disc space between two vertebrae.

FIG. 10 is a perspective view of the guide mechanism and milling mechanism assembly of the present invention showing removal of the milling mechanism from a disc space between two vertebrae.

FIG. 11 is a sectioned view of the guide mechanism and milling mechanism assembly of the present invention showing guided insertion into a disc space between two vertebrae.

FIG. 12 is a sectioned view of the guide mechanism and milling mechanism assembly of the present invention shown after insertion into a disc space between two vertebrae.

FIG. 13 is a sectioned view of the guide mechanism and milling mechanism assembly of the present invention showing removal of the milling mechanism from a disc space between two vertebrae and the space created by the milling process.

FIG. 14 is an alternative embodiment of the guide mechanism.

FIG. 15a is a further alternative embodiment of the guide mechanism.

FIG. 15b is a further alternative embodiment of the guide mechanism.

FIG. 16a shows a “disc replacement prosthesis” being inserted into a cavity formed with the invention.

FIG. 16b shows a “disc replacement prosthesis” after insertion into a cavity formed with the invention.

FIG. 17a shows a threaded fusion cage being inserted into a cavity formed with the invention.

FIG. 17b shows a threaded fusion cage after insertion into a cavity formed with the invention.

FIG. 18a shows an alternative embodiment of the counterbore portion of the invention comprising a flexure.

FIG. 18b shows a further alternative embodiment of the counterbore portion of the invention comprising a bellows.

FIG. 19a shows an exploded perspective view of an alternative embodiment of the mill portion of the invention incorporating a removable counterbore.

FIG. 19b shows a perspective view of an alternative embodiment of the mill portion of the invention incorporating a removable counterbore.

FIG. 20 shows a perspective view of an alternative embodiment of the mill portion of the invention incorporating a non-removable counterbore.

### DETAILED DESCRIPTION OF THE PREFERRED EMBODIMENTS

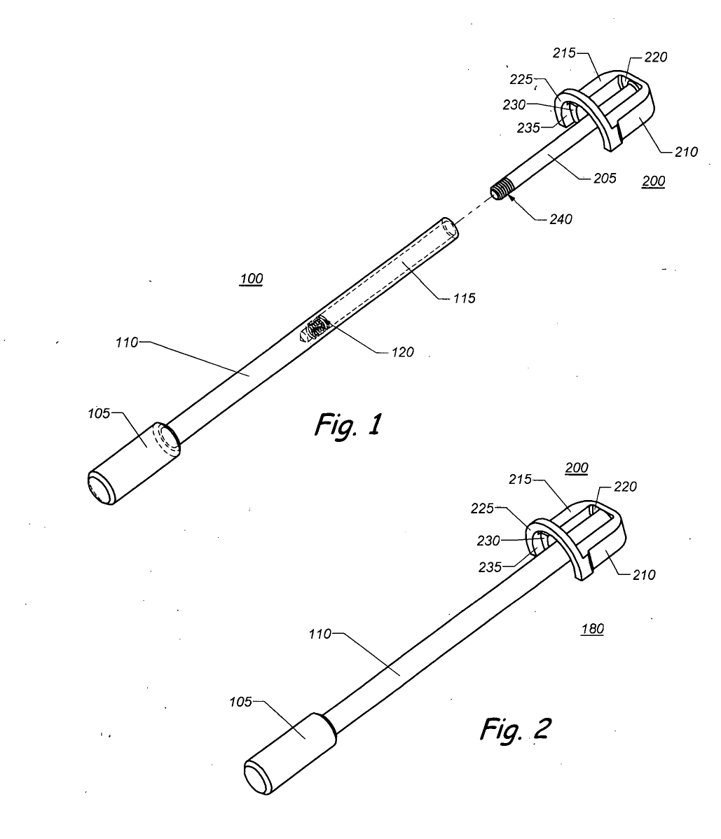

FIGS. 1 & 2 show a guide mechanism 200 and an insertion handle 100. Insertion handle 100 consists of an elongated shaft 110 with a blind hole 115 and a gripping handle 105. At the bottom of blind hole 115 is a threaded portion 120 for attachment to guide mechanism 200. Guide mechanism 200 consists of an intervertebral spacer 210, an elongated guide shaft 205 and a depth stop 225. On the end of elongated guide shaft 205 is a threaded portion 240 for attachment to threaded portion 120 of insertion handle 100. Depth stop 225 has a circular cut-out 235 creating a shoulder 230 for stopping a counterbore which will be described below. Intervertebral spacer 210 consists of sidewalls 215 and rear rim 220. FIG. 1 is an exploded view to show the separate parts and FIG. 2 shows the assembly. It is anticipated that multiple sizes of guide mechanism 200 will be necessary to ensure a proper fit for a particular size of disc space 20 which will be described in the following figures.

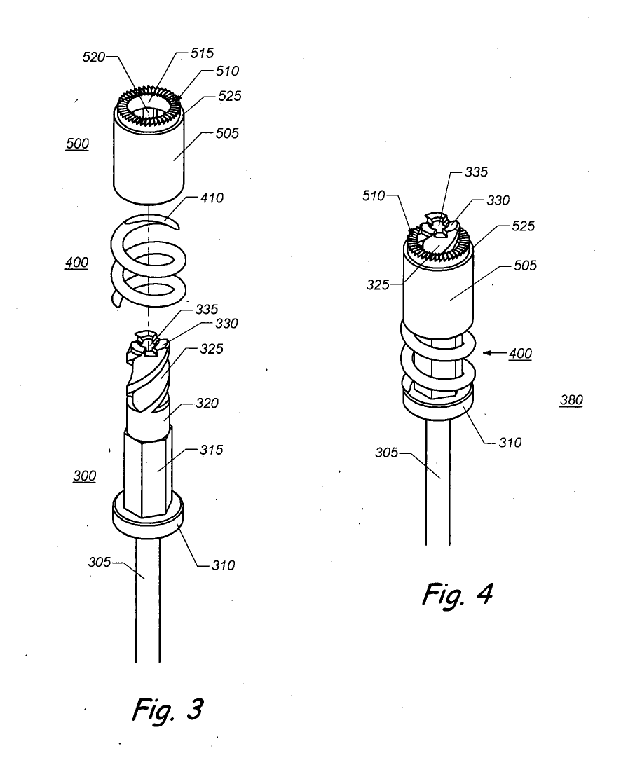

FIGS. 3 & 4 show a milling mechanism assembly 380 which consists of a counterbore 500, a compression spring 400 and a mill 300. Mill 300 has an elongated shaft 305, a shoulder 310, a hex portion 315 and a cutting portion 320. Cutting portion 320 has cutting flutes 325, an end portion 330 and a blind hole 335. In this preferred embodiment, end portion 330 has features for end cutting, but this may not be necessary. Spring 400 has flat ends 410 so that spring 400 rests on shoulder 310 of mill 300. Counterbore 500 contains cutting teeth 510, an elongated body 505, a through hole 515, a shoulder 525, and a hex portion 520 which engages hex portion 315 so that counterbore 500 spins in synchronization with mill 300. FIG. 3 is an exploded view to show the separate parts and FIG. 4 shows the assembly. It is anticipated that multiple sizes of milling mechanism assembly 380 will be necessary to ensure a proper fit for a particular size of disc space 20 which will be described in the following figures.

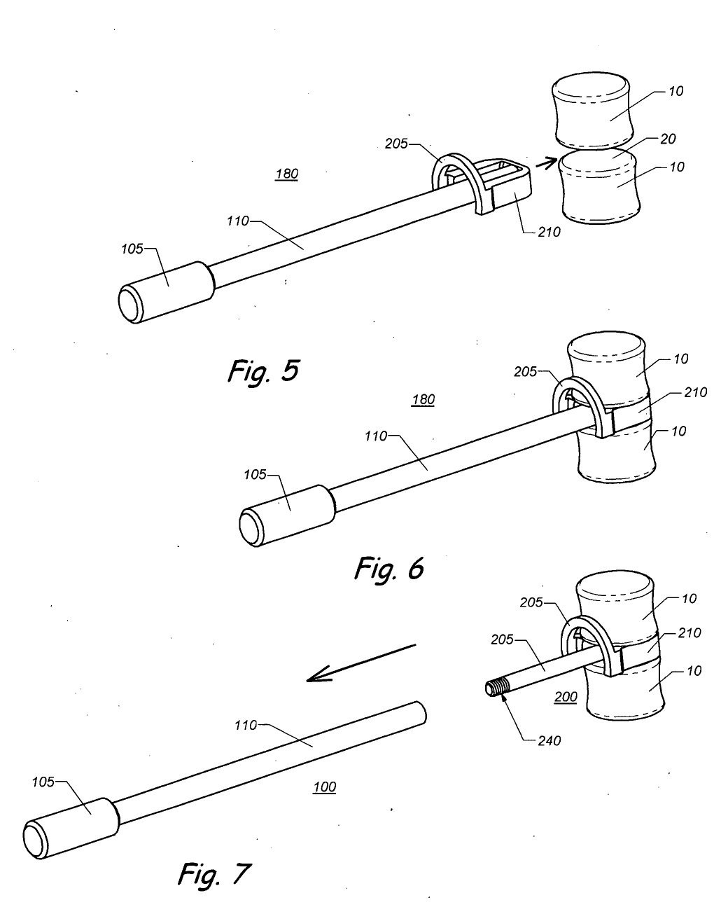

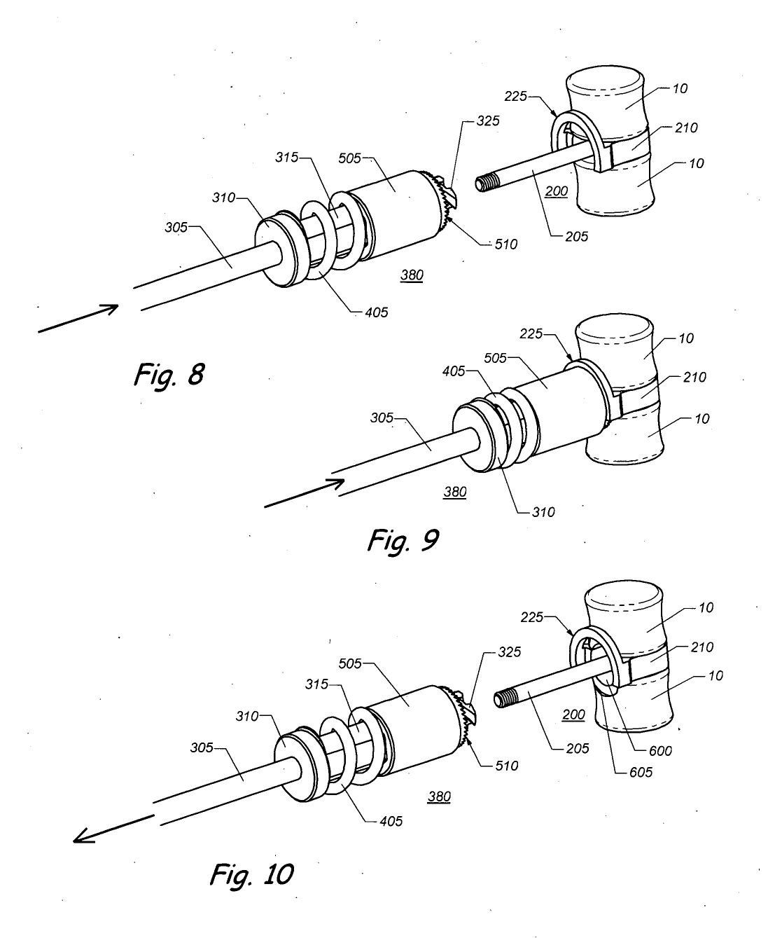

FIG. 5, 6 & 7 show a method of inserting guide mechanism 200 into a disc space 20 between two adjacent vertebrae 10. Guide mechanism and insertion handle assembly 180 is inserted into disc space 20 which has been prepared by the removal of some or all of the intervertebral disc material. Typically, the annulus fibrosis will be left intact on the lateral portions of disc space 20, but this material is not shown in the figures for clarity. Guide mechanism and insertion handle assembly 180 is pushed, tapped, vibrated or otherwise inserted into disc space 20. FIG. 6 shows Guide mechanism and insertion handle assembly 180 inserted into disc space 20. When the placement and orientation of guide mechanism 200 in disc space 20 has been satisfactorily achieved, insertion handle 100 is removed by unthreading it from threaded portion 240 on guide mechanism 200 and withdrawal from the surgical site. This is shown in FIG. 7. After removal of insertion handle 100, milling mechanism assembly 380 is chucked into a drill (not pictured: either a manual drill or a power drill utilizing an electric motor or pneumatic motor) and is mounted onto guide mechanism 200 by sliding elongated guide shaft 205 into blind hole 335 in mill 300 as shown in FIG. 8. In this way, milling mechanism assembly 380 is guided through a precise path to cut a precise circular recess 600 into adjacent vertebrae 10. Milling mechanism assembly 380 is further inserted into disc space 20 until elongated guide shaft 205 bottoms out in blind hole 335 as shown in FIG. 9. During this process, counterbore 500 cuts a circular shoulder 605 into the front of adjacent vertebrae 10. The depth of circular shoulder 605 is determined by the engagement of shoulder 525 onto depth stop 225. Spring 400 compresses to allow full insertion of mill 300 into guide mechanism 200. Therefore, the same milling mechanism assembly 380 can be used to make a variety of hole depths and can therefore be used with multiple sizes of guide mechanism 200. After completing the boring operation, milling mechanism assembly 380 is withdrawn from guide mechanism 200 as shown in FIG. 10. The diameter of elongated guide shaft 205 will be matched with blind hole 335, the diameters of which may be varied to correspond with different heights of intervertebral spacer 210 so as to avoid incorrect boring of circular recess 600 and circular shoulder 610 by not allowing blind hole 335 to engage incorrect sizes of elongated guide shaft 205.

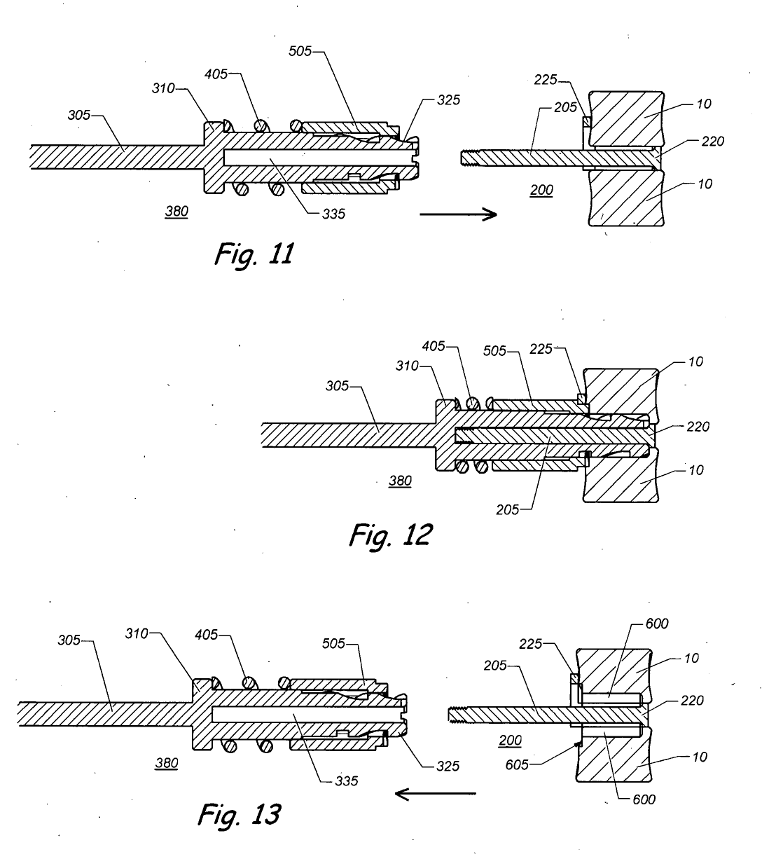

FIGS. 11, 12 & 13 show the same steps as described above, but in sectioned views to illustrate the creation of circular recess 600 and circular shoulder 605.

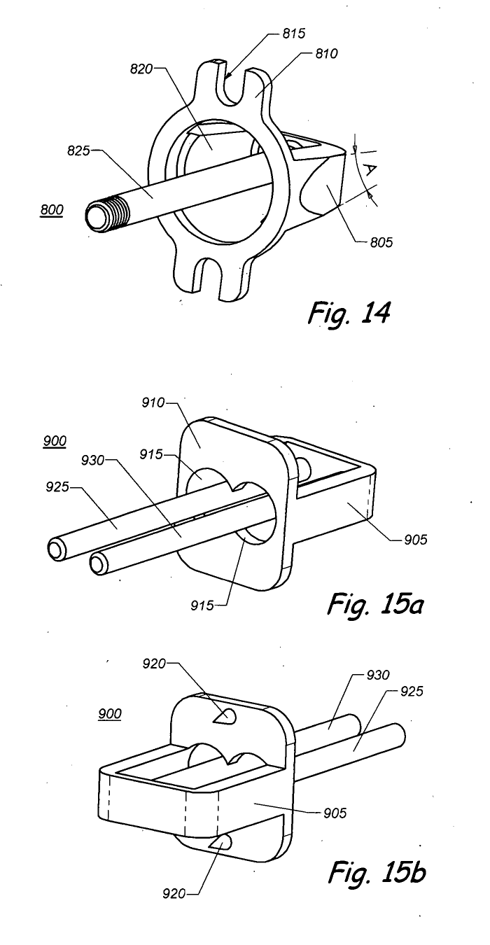

FIG. 14 shows an alternative guide mechanism embodiment 800 which incorporates an angular intervertebral portion 805 with an angle A which is meant to match a lordotic angle in disc space 20. In addition, alternative guide mechanism 800 incorporates a tapered bore 820 to accept a tapered mill. An alternative depth stop 810 with slots 815 is meant to allow either attachment to adjacent vertebrae 10 with screws or pins or to slide over a Caspar type distractor or other distractor means.

FIGS. 15a & 15b show a further alternative guide mechanism embodiment 900 which incorporates two elongated guide shafts 925 and 930 for guiding mill 300 to create two circular recesses for placement of two adjacent implants. More than two elongated guide shafts can be implemented for implantation of more than two implants. The orientation of elongated guide shafts 925 and 930 may be varied to create circular recesses and/or circular shoulders with differing orientations. FIG. 15b is a back view of further alternative guide mechanism embodiment 900 to illustrate attachment means 920 which may be in the form of spikes for firm attachment to adjacent vertebrae 10.

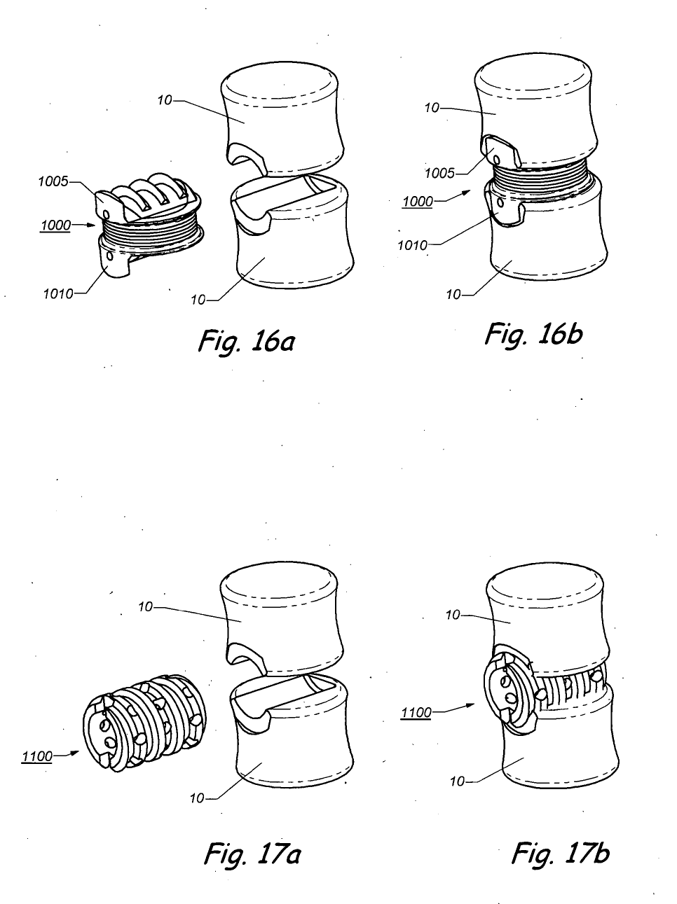

FIGS. 16a & 16b illustrate implantation of a disc replacement prosthesis 1000 into disc space 20. Disc replacement prosthesis 1000 incorporates tabs 1005 & 1010 which fit into circular shoulders 605. FIG. 16a shows disc replacement prosthesis 1000 before implantation and FIG. 16b shows the completed implantation.

FIG. 17a & 17b illustrate implantation of a fusion cage 1100 into disc space 20. Circular shoulders 605 may or may not be necessary for this case, and counterbore 500 may therefore be eliminated from milling mechanism assembly 380 if necessary. FIG. 17a shows implant 1100 before implantation and FIG. 17b shows the completed implantation.

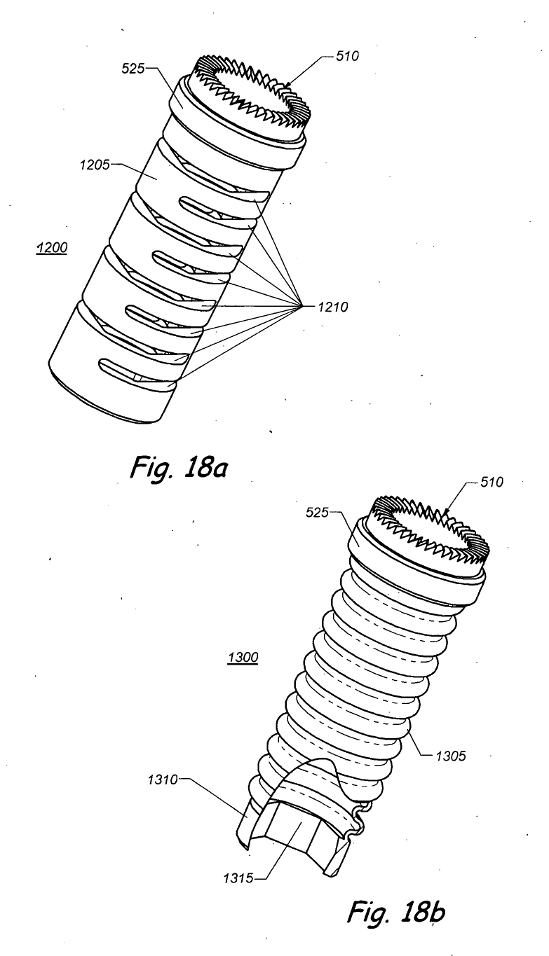

FIG. 18a shows an alternative counterbore 1200 embodiment where spring 400 is incorporated into the counterbore by the addition of flexure slots 1210 into body 1205 to allow compliance of alternative counterbore 1200 to allow mill 300 to bore a proper hole depth. Alternative counterbore 1200 has a hex shaped bore to engage hex portion 315 on mill 300. FIG. 18b shows a further alternative counterbore 1300 embodiment which is a bellows type construction with convolutions 1305 to allow compliance of further alternative counterbore 1300 to allow mill 300 to bore a proper hole depth. A cut away view is included in the figure to illustrate convolutions 1305 and to show hex portion 1315 which engage hex portion 315 on mill 300.

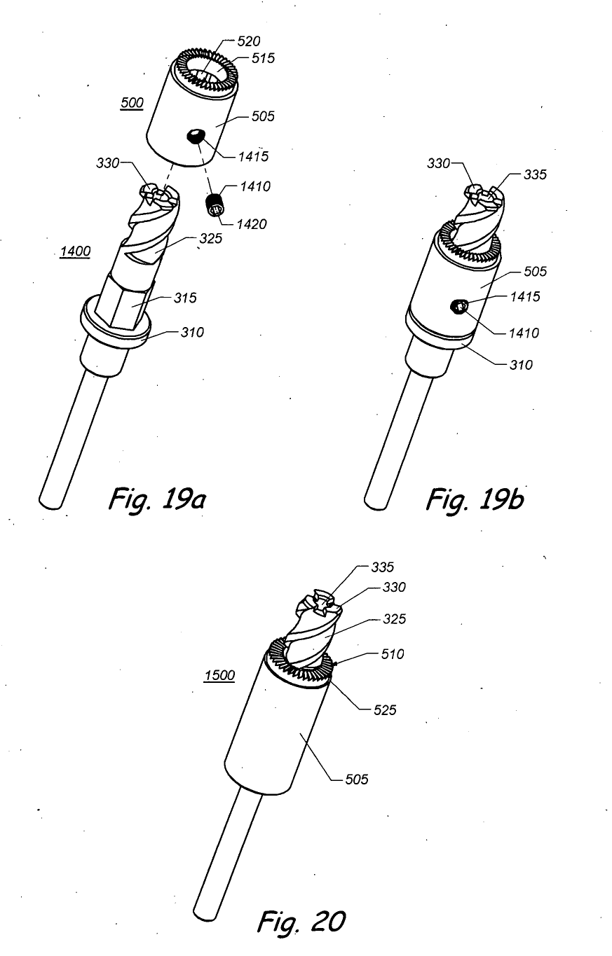

FIG. 19a & 19b show an alternative counterbore embodiment which does not utilize a spring. In this embodiment, counterbore 500 utilizes a set screw 1420 for attachment to mill 1400. FIG. 19a is an exploded view to show the individual pieces, FIG. 19b shows the assembly.

FIG. 20 shows another alternative embodiment where the mill and counterbore have been consolidated into one combination mill 1500.

1. A device for creation of a cavity between two bone surfaces, comprising:
a. a spacing mechanism for placement between said bone surfaces;
b. one or more guide mechanisms disposed on said spacing mechanism within said cavity for controlling a bone cutting device.

2. The device of claim 1, further incorporating a means for controlling the placement of the device by abutting one or more of the bone surfaces

3. The device of claim 1, whereby the cavity created is between the bone surfaces

4. The device of claim 1, whereby the cavity created is partially overlapping the bone surfaces and within the body of said bone

5. The device of claim 1, wherein said spacing mechanism is sized to fit within and maintain a fixed distance between said bone surfaces

6. The device of claim 1, wherein said bone cutting device is placed over said guide mechanism

7. The device of claim 1, wherein said spacing mechanism provides a depth stop to said bone cutting device

8. The device of claim 1, wherein said guide mechanism provides a depth stop to said bone cutting device

9. The device of claim 1, wherein said cavity in each bone is created with one or more passes of said bone cutting device

10. The device of claim 1, wherein said bone cutting device creates a counterbore feature on and within one or more of the bones

11. A device for creation of a cavity at least partially within two adjacent bones, comprising:
a. a spacing mechanism for placement between said bone surfaces;
b. one or more guide mechanisms disposed on said spacing mechanism within said cavity for controlling a bone cutting device.

12. The device of claim 11, further incorporating a means for controlling the placement of the device by abutting one or more of the bone surfaces

13. The device of claim 11, whereby the cavity created is partially overlapping the bone surfaces and within the body of said bone

14. The device of claim 11, wherein said spacing mechanism is sized to fit within and maintain a fixed distance between said bone surfaces

15. The device of claim 11, wherein said bone cutting device is placed over said guide mechanism

16. The device of claim 11, wherein said spacing mechanism provides a depth stop to said bone cutting device

17. The device of claim 11, wherein said guide mechanism provides a depth stop to said bone cutting device

18. The device of claim 11, wherein said cavity in each bone is created with one or more passes of said bone cutting device

19. The device of claim 11, wherein said bone cutting device creates a counterbore feature on and within one or more of the bones
